(19) United States
(12) Patent Application Publication
(10) Pub. No.: US 2025/0099658 A1
(43) Pub. Date: Mar. 27, 2025

(54) BLOOD TREATMENT APPARATUS WITH INTEGRITY TEST FOR A DIALYSIS LIQUID SYSTEM

(71) Applicant: Fresenius Medical Care Deutschland GmbH, Bad Homburg (DE)

(72) Inventors: Peter Kloeffel, Bad Homburg (DE); Tobias Irrgang, Bad Homburg (DE)

(21) Appl. No.: 18/834,083

(22) PCT Filed: Jan. 30, 2023
(86) PCT No.: PCT/EP2023/052148

§ 371 Date: Jul. 29, 2024

(30) Foreign Application Priority Data
Jan. 31, 2022 (DE) 10 2022 102 141.6

(51) Int. Cl. A61M 1/16; G16H 40/63

(52) U.S. Cl. CPC A61M 1/1656; G16H 40/63; A61M 2205/3327; A61M 2205/3331; A61M 2205/50

(57) ABSTRACT

A blood treatment apparatus comprises a dialysis liquid system having devices for mixing a dialysis liquid from, or with, at least one first concentrate from a concentrate supply system. The blood treatment apparatus comprises a first connecting line arranged upstream of the dialysis liquid system having a first connector for a fluidic connection of a section of the dialysis liquid system to the concentrate supply system. The blood treatment apparatus further comprises a first valve, which is provided downstream of the first connector in or on the first connecting line. The blood treatment apparatus further comprises a control device or closed-loop control device.

### CROSS-REFERENCE TO RELATED APPLICATIONS

The present application is the national stage entry of International Patent Application No. PCT/EP2023/052148, filed on Jan. 30, 2023, and claims priority to Application No. DE102022102141.6 filed in the Federal Republic of Germany on Jan. 31, 2022, the disclosures of which are expressly incorporated herein in their entirety by reference thereto.

### TECHNICAL FIELD

The disclosure relates to a blood treatment apparatus according to claim and a control device or closed-loop control device according to claim 14 or according to the preambles or generic terms of these claims.

### BACKGROUND

Various types of blood treatment apparatuses are known from practice. Included among them are e.g. apparatuses for hemodialysis, hemofiltration and hemodiafiltration. During the extracorporeal blood treatment, blood flows in an extracorporeal blood circuit through a blood treatment unit. In the apparatuses for hemodialysis, hemofiltration and hemodiafiltration, the blood treatment unit is a dialyzer or blood filter which, in simple terms, is separated into a blood chamber and a dialysis liquid chamber by a semipermeable membrane. During the blood treatment by hemodialysis or hemodiafiltration, the blood flows through the blood chamber, while a dialysis liquid flows through the dialysis liquid chamber.

Unspent dialysis liquid flows into the dialysis liquid chamber, usually in countercurrent to the direction in which the blood flows through the blood chamber, and then leaves, now considered spent, the dialyzer as dialysate. In some applications, the dialysis liquid used for this purpose is automatically mixed from concentrates by the blood treatment apparatus, for which purpose the blood treatment apparatus is connected, to a concentrate supply system, which is external to the blood treatment apparatus e.g. on the hospital side. Corresponding connectors and/or connecting lines are provided for this purpose.

The description below relates to a blood treatment apparatus and a control device or closed-loop control device for a blood treatment apparatus.

### SUMMARY

The disclosure relates to a blood treatment apparatus which comprises a dialysis liquid system having, or consisting of, devices for mixing dialysis liquid from or with at least a first concentrate which may be supplied to the blood treatment apparatus from a concentrate supply system which is not included in the blood treatment apparatus. These devices may include, for example, a bicarbonate pump and/or a sodium pump, as well as associated lines and the like.

Further, the blood treatment apparatus described herein comprises a first connecting line being arranged upstream of the dialysis liquid system or of the devices for mixing a dialysis liquid and having a first connector. The first connector is used to fluidically connect a section of the dialysis liquid system to the concentrate supply system. In the concentrate supply system, for example, bicarbonate and sodium may be present in a concentrated manner and may be supplied in a suitable dose to the mixing devices mentioned herein, which form part of the dialysis liquid system, via the first connecting line connected to the dialysis liquid system by the first connector. In this way, a dialysis liquid, preferably specifically tailored to the treatment of a particular patient, may be generated.

The blood treatment apparatus further comprises a first valve provided downstream of the first connector in or on the first connecting line. The first valve serves to allow or prevent a flow through the first connecting line.

Furthermore, a control device or closed-loop control device is disclosed herein.

Embodiments may comprise some, several or all of the following features in any combination, unless the person skilled in the art recognizes this combination as technically impossible.

In all of the statements made herein, the use of the expression “may/can be” or “may/can have” and so on, is to be understood synonymously with “preferably is” or “preferably has,” and so on respectively, and is intended to illustrate embodiments.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” (also “a/an”) as encompassing “at least one”. This understanding is also equally encompassed by the present description as the interpretation that a numeric word, for example, “one” (also “a/an”) may alternatively mean “exactly one”, wherever this is evidently technically possible for the person skilled in the art. Both understandings are encompassed by the present description and apply herein to all used numerical words.

Whenever reference is made herein to spatial indications, such as “top”, “bottom”, “left” or “right”, the skilled person understands this to mean the arrangement in the figures attached hereto and/or in the state of use. “Bottom” is closer to the center of the earth or the lower edge of the figure than “top”.

Whenever “downstream” is mentioned herein, this is to be understood as “into the blood treatment apparatus”, i.e., in particular from the concentrate supply system towards the dialysis liquid system.

Advantageous developments of certain embodiments include the subject-matter of the dependent claims and corresponding embodiments.

Whenever an embodiment is mentioned herein, it represents an exemplary embodiment.

When it is disclosed herein that the subject-matter according to the present description comprises one or several features in a certain embodiment, it is also respectively disclosed herein that the subject-matter according to the present description does, in other embodiments, likewise according to the present description, explicitly not comprise this or these features, for example, in the sense of a disclaimer. Therefore, for every embodiment mentioned herein it applies that the converse embodiment, e.g. formulated as negation, is also disclosed.

When method steps are mentioned herein, then the blood treatment apparatus according to the present description or the control device or closed-loop control device according to the present description is in several embodiments configured in order to execute, in any combination, one or several of these method steps, in particular when these are automatically executable steps, or in order to accordingly control corresponding apparatuses which preferably correspond with their names to the designation of the respective method step (for example, “determining” as a method step and “apparatus for determining” for the apparatus) and which may likewise be part of the apparatus(es) according to the present description or may be connected thereto in signal communication.

When programmed or configured is mentioned herein, then these terms may in some embodiments be interchangeable.

When a signal communication or communication connection between two components is mentioned herein, this may be understood to mean a connection that exits during use. It may also be understood that a preparation for such a (wired, wireless or otherwise implemented) signal communication exists, for example by coupling both components, for example by pairing, etc.

Pairing is a process that takes place in connection with computer networks in order to establish an initial link between computer units for the purpose of communication. The best-known example of this is the establishing of a Bluetooth connection, by which various devices (e.g. smartphone, headphones) are connected to each other. Pairing is sometimes also referred to as bonding.

A control device or closed-loop control device may prompt the execution of all or substantially all of the method steps. A method disclosed herein may substantially or completely be executed by the control device or closed-loop control device. It may be partly executed by the control device or closed-loop control device, in particular those method steps may be executed by the control device or closed-loop control device that do not require or involve human intervention and/or preparation.

In several embodiments, the control device or closed-loop control device is present in or on the blood treatment apparatus, for instance together with other components or devices of the blood treatment apparatus in a common housing of the blood treatment apparatus.

In some embodiments, the blood treatment apparatus further comprises a first pressure measuring device, which serves to measure a pressure prevailing in the first connecting line. Here, the first pressure measuring device is arranged, or measures, between the first connector and the first valve. The first valve is preferably the valve closest downstream to the first connector along the first connecting line.

In several embodiments, the blood treatment apparatus further comprises a second connecting line which is arranged upstream of the dialysis liquid system or of the devices for mixing dialysis liquid and having a second connector. The second connector serves to fluidically connect the dialysis liquid system to the concentrate supply system via the second connecting line. The second connecting line may therefore be seen as analogous to the first connecting line and may extend for example parallel to the latter.

In these embodiments, the blood treatment apparatus further comprises a second valve, which is provided downstream of the second connector in or on the second connecting line.

In some embodiments, the blood treatment apparatus further comprises a second pressure measuring device, which serves to measure a pressure prevailing in the second connecting line. In this, the second pressure measuring device is arranged between the second connector and the second valve or measures there. The second valve is preferably the valve closest to the second connector along the second connecting line.

In several embodiments of the blood treatment apparatus, the first and/or the second connecting line (each) does not comprise, or is not fluidically connected to, a ventilation valve for establishing a connection to the atmosphere. Alternatively, if they have such ventilation valves, the control device or closed-loop control device in these embodiments is not programmed to allow a connection to the atmosphere via them as part of a function test or tightness test of the valve, and preferably in order not to effect a connection to the atmosphere at all.

The first and/or the second connecting line thus preferably does not have any valves which establish or can establish a connection to the atmosphere downstream of the first or second connector, respectively, and/or which would be actuated for this purpose by the control device or closed-loop control device, e.g. as part of a function test.

In some embodiments, the blood treatment apparatus further comprises a third pressure measuring device. The third pressure measuring device is provided in the dialysis liquid system. It is suitable and provided for measuring a pressure prevailing in the aforementioned section of the dialysis liquid system, wherein the section is present downstream of the first connecting line and/or the second connecting line. The section is in fluid communication with the first connecting line and/or the second connecting line up to the first or the second valve, but not across the latter when the first or the second valve, respectively, is closed. Sections of the first connecting line and/or the second connecting line which are arranged upstream of the first or second valve, respectively, are in fluid communication with the section only when the first or second valve, respectively, is open. However, during the pressure measurements described herein, the first and/or the second valve is preferably closed for testing the tightness of the first or the second valve, respectively.

In order to be able to keep the section closed during a pressure measurement, or to be able to close it for the pressure measurement, the blood treatment apparatus comprises in several embodiments suitably arranged shut-off devices. The shut-off devices thus serve to fluidically close the section, in particular with respect to some or to all of the other sections of the dialysis liquid system, or may be used for this purpose. If required, they are actuated accordingly by the control device or closed-loop control device. The shut-off devices may above all be or comprise valves, but are not limited thereto. For example, occluding pumps or clamps may also be counted among the shut-off devices. In particular, the shut-off devices include the first valve and/or the second valve.

In several embodiments of the blood treatment apparatus or of the control device or closed-loop control device, it further comprises an evaluation unit. The evaluation unit is configured to evaluate pressures in the section and/or in the connecting lines being measured by the first, second and/or third pressure measuring device. Alternatively or additionally, the evaluation unit is configured to evaluate a pressure difference. In this case, said difference may have been measured between successive pressure measurements which were made by one and the same pressure measuring device, e.g. by the first, by the second or by the third pressure measuring device. The difference may additionally or alternatively have been determined between at least two different pressure measuring devices, e.g. the first and the third pressure measuring device.

The evaluation may be or encompass a comparison to thresholds, limits, ranges, criteria, etc.

In some embodiments, the blood treatment apparatus further comprises a conveying device which is in conveying relationship with the section of the dialysis liquid system which in turn is in fluid communication, when the first and/or second valve is open, with the first connecting line and/or the second connecting line.

In several embodiments, the blood treatment apparatus further comprises a control device or closed-loop control device configured to prompt the following steps or method steps:
a) building up or setting a, preferably predetermined, overpressure or underpressure as a first test pressure in the section which is closed for the pressure measurement and being e.g. completely filled with fluid, e.g. liquid, by using the conveying device.
b) measuring, in a first pressure measurement, the pressure prevailing in the section using the third pressure measuring device, the pressure prevailing in the first connecting line using the first pressure measuring device and/or the pressure prevailing in the second connecting line using the second pressure measuring device.

In some embodiments, the control device or closed-loop control device is further configured to carry out the first pressure measurement using the first, second or third pressure measuring device at a time after the build-up or setting of the, preferably predetermined, first test pressure in the closed section and to have the pressure measured thereby evaluated by the evaluation unit. Also in this embodiment, the closed section is, for example, completely filled with fluid, e.g. liquid. The overpressure or underpressure is also built up or set, for example, by the conveying device.

In several embodiments, this first pressure measurement follows the build-up or setting of the pressure in accordance with method step a). Preferably, there is a predetermined minimum and/or maximum time period between method step a) and the measurement from method step b), which must at least have elapsed or may at most have elapsed after the pressure has been built up or set, before the first pressure measurement takes place.

In several embodiments, the control device or closed-loop control device is further configured to carry out the first pressure measurement using the first and/or the second pressure measuring device(s) at a time prior to the build-up or setting of the, preferably predetermined, first test pressure in the closed section, and to define the pressure measured thereby as initial pressure. Furthermore, the control device or closed-loop control device is configured in these embodiments to carry out a second pressure measurement using the pressure measuring device with which the first pressure measurement was carried out, at a time after the build-up or setting of as, preferably predetermined, first test pressure in the closed section and to define the pressure measured thereby as end pressure. In these embodiments, the control device or closed-loop control device is further configured to have a pressure difference between the initial pressure and the end pressure evaluated by the evaluation unit.

The conditions for filling the section and for building up or setting the overpressure or underpressure, respectively, preferably also apply as explained herein to this embodiment.

In some embodiments, the evaluation unit is part of the control device or closed-loop control device.

In several embodiments, the control device or closed-loop control device of the blood treatment apparatus is further configured to prompt the following method steps:
c) building up or setting at least a, preferably predetermined, overpressure or underpressure, which differs from the overpressure or underpressure of the first test pressure, as second test pressure or again further test pressure in the section by the conveying device;
d) measuring, e.g. in a second pressure measurement, the pressure prevailing in the section using the third pressure measuring device after the second test pressure has been built up or set; and
e) evaluating both the pressure measured during the first pressure measurement and during the second pressure measurement using the evaluation unit, wherein suitable pressure differences may also be evaluated here instead of pressures, for example between the second test pressure and the result of the second pressure measurement.

In some embodiments, the control device or closed-loop control device is further configured to carry out the performed method steps from the group consisting of method steps a) to e), alternatively method steps b) and/or d), without establishing or causing, for this purpose, a connection between the first connecting line and/or the second connecting line with the atmosphere by corresponding valve switching.

In several embodiments, the blood treatment apparatus is embodied as a dialysis apparatus, hemodialysis apparatus, hemofiltration apparatus or hemodiafiltration apparatus, in particular as an apparatus for the acute renal replacement therapy, the chronic renal replacement therapy or for the continuous renal replacement therapy (CRRT).

In several embodiments, the first, second, or further test pressure is not known as pressure in terms of its magnitude measurable by a pressure measuring device (e.g., in the units hectopascal [hPa]). Rather, in such embodiments, it is provided to build up a pressure which, in the case of a section filled with e.g. liquid, results from the fact that the conveying device conveys for a certain duration, makes a certain number of revolutions or conveying movements, or similar. Knowledge of the exact level of the pressure built up is therefore not necessary in some embodiments.

Some or all of the embodiments according to the present description may have one, several, or all of the advantages listed above and/or below.

The design of the connecting line according to the present description for connecting the blood treatment apparatus to the concentrate supply system makes it possible to suffice or manage preferably with only one valve or with only two valves between the first or second connector and e.g. the dialysis liquid system or its devices for mixing the dialysis liquid, or with the pressure measuring device referred to herein as the third pressure measuring device. Further valves can advantageously be dispensed with, in particular in the area of the connecting line(s), especially for establishing a connection to the atmosphere.

It is thus possible by the design according to the present description to manage without a ventilation valve or ventilation pot in the first and/or second connecting line, which would otherwise be necessary in order to ensure that a pressure gradient can be built up across the valves to be tested, which is sufficient to detect leaks across the respective valve, when testing the connection for the concentrate supply system as part of a tightness test for the first and/or for the second valve.

If the designs according to the present description described herein were to be omitted, a ventilation valve would be required on the side of the two valves facing the concentrate supply system, which may ensure a known pressure at the time of the test, in this case atmospheric pressure.

However, if there were such a ventilation valve and if the valves were consequently tested against the atmosphere and thus against the known atmospheric pressure, the concentrate present in the concentrate supply system or in one of the connecting lines could crystallize on contact with air. As a result, the ventilation valves (and their associated pots) would have to be replaced regularly, as addressed above, since they would regularly become encrusted and clogged, as a result of which their ventilation function would no longer be ensured. Certain embodiments can dispense with such ventilation valves, and therefore the above-described problem is avoided. Thus, time and costs can be saved by means of such embodiments.

According to the present description, it can rather be ensured, especially by the first pressure measuring device in the first connecting line (or the second pressure measuring device in the second connecting line) on the one hand and the third pressure measuring device in the dialysis liquid system on the other hand, that the pressure currently prevailing in the concentrate supply system does not coincidentally correspond to the test pressure built up in the dialysis liquid system by the conveying device. If this could not be ruled out, then it could not be ruled out that no pressure gradient would be established across the valves to be tested. Since no volume can be moved without such a pressure gradient, eventual valve leakage would also not be detectable. In some embodiments the test pressure may be set as a function of the current pressure prevailing in the concentrate supply system, in particular deviating therefrom, which may be measured by the first or second pressure measuring device. If, as is the case in some embodiments, no ventilation valves are provided, the tightness of the relevant valves may be checked by underpressure as a test pressure, or by applying different test pressures. Corresponding programming of the control device or closed-loop control device may thus also make it possible to dispense with the first or second pressure measuring device in the connecting lines.

### BRIEF DESCRIPTION OF THE DRAWINGS

In the following, the techniques described herein are purely exemplarily described with reference to the accompanying figures, in which the same reference numerals denote identical or similar components. The following applies:

FIG. 1 shows sections of a flow diagram of a dialysis liquid system of a blood treatment apparatus in a first embodiment;

FIG. 2a shows the flow diagram of the dialysis liquid system of FIG. 1 with a highlighted section;

FIG. 2b shows the flow diagram of the dialysis liquid system analogous to that of FIG. 1 in a further embodiment of the blood treatment apparatus;

FIG. 3 shows, schematically highly simplified, the closed section of a blood treatment apparatus in a further embodiment;

FIG. 4a shows, schematically highly simplified, the method step of measuring b) or measuring d), respectively, carried out in a first manner using the blood treatment apparatus;

FIG. 4b shows, schematically highly simplified, the method step of measuring b) carried out in a further manner using the blood treatment apparatus with a tight valve;

FIG. 4c shows, schematically highly simplified, the method step of measuring b) of FIG. 4b with a possibly leaking valve; and

FIG. 4d shows, schematically highly simplified, the method step of measuring b) carried out in a third manner using the blood treatment apparatus.

### DETAILED DESCRIPTION

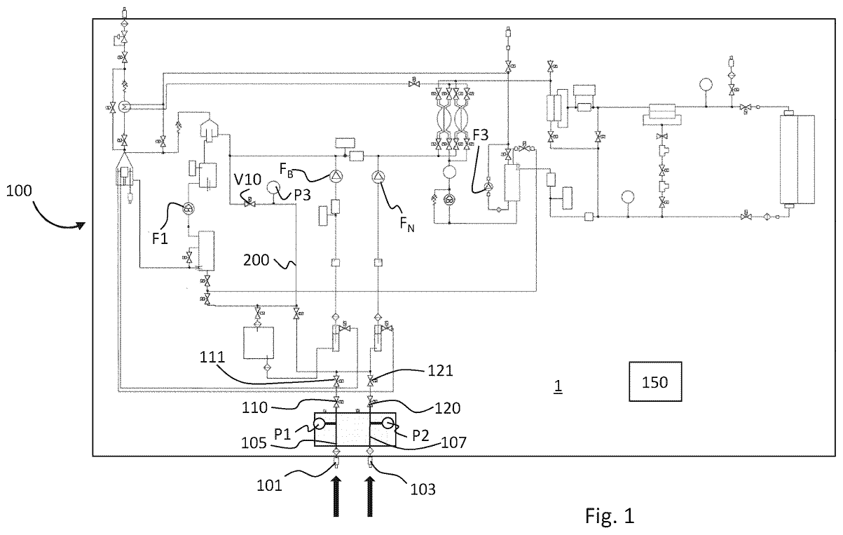

FIG. 1 shows sections of a flow diagram of a dialysis liquid system 1 (also referred to as hydraulics) of a blood treatment apparatus 100 in a first embodiment.

The dialysis liquid system 1 comprises a plurality of pumps, valves, actuators, sensors and further components, such as conveying devices. They may all be independently in signal communication with the control device or closed-loop control device 150 and be optionally controlled and/or read out by it.

In the example of FIG. 1, the dialysis liquid system 1 comprises, for example, devices for mixing a dialysis liquid from or with at least one concentrate. These devices may, among others, include for example the bicarbonate pump F_{B} and the sodium pump F_{N} from the series of pumps mentioned above, as well as associated lines and the like.

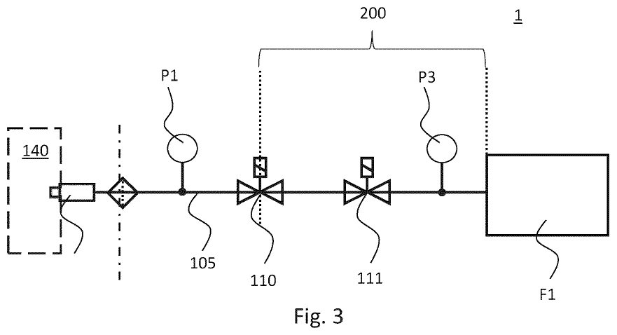

The blood treatment apparatus 100 further comprises a first connecting line 105 with a first connector 101 arranged upstream of the dialysis liquid system 1 or upstream of the devices for mixing dialysis liquid, wherein the flow direction is assumed into the dialysis liquid system 1 from upstream to downstream, as indicated by arrows at the bottom edge of FIG. 1, a flow direction which is present during operation of the dialysis liquid system 1 during operation of the blood treatment apparatus 100. The first connector 101 serves for the fluidic connection of the dialysis liquid system 1 or a section 200 of the dialysis liquid system 1 to a concentrate supply system 140 (not shown in FIG. 1, see FIG. 3 for details).

A first valve 110 provided downstream of the first connector 101 in or on the first connecting line 105 is also comprised by the blood treatment apparatus 100, optionally also a first pressure measuring device P1 for measuring a pressure prevailing in the first connecting line 105. The first pressure measuring device P1 is, if exemplarily provided as in FIG. 1, for this purpose arranged between the first connector 101 and the first valve 110 or measures there. The first valve 110 is the valve closest to the first connector 101 along the first connecting line 105.

The blood treatment apparatus 100 further comprises a second connecting line 107 with a second connector 103 arranged upstream of the dialysis liquid system 1 or upstream of the devices for mixing the dialysis liquid. This second connector 103 also serves for the fluidic connection of the dialysis liquid system 1 to the concentrate supply system 140.

A second valve 120, which is provided downstream of the second connector 103 in or on the second connecting line 107, is also comprised by the blood treatment apparatus 100, as well as an optional second pressure measuring device P2 for measuring a pressure prevailing in the second connecting line 107. The second pressure measuring device P1 is, if exemplarily provided as in FIG. 1, for this purpose arranged between the second connector 103 and the second valve 120 or measures there. The second valve 120 is the valve closest to the second connector 103 along the second connecting line 107.

The blood treatment apparatus 100 further comprises a conveying device designated with F1, here for example the ultrafiltration pump of the hydraulic system. The conveying device may comprise only the pump designated with F1, or may comprise different pumps.

FIG. 1 further shows a third pressure measuring device P3. This serves to measure a pressure prevailing in the section 200 of the dialysis liquid system 1. The section 200 is arranged downstream of the first valve 110 of the first connecting line 105 and/or downstream of the second valve 120 of the second connecting line 107 or is connected to one or both of these valves 110, 120, preferably directly, as shown in FIG. 1. In this, the section 200 is in fluid communication with the first connecting line 105 and/or the second connecting line 107. However, a material exchange between section 200 and first connecting line 105 and/or second connecting line 107 presupposes that the first valve 110 or the second valve 120, respectively, is not closed.

A further, optional valve which is arranged downstream of the first connecting line 105 and can also close the section 200 against the first connecting line 105, is denoted with 111 in FIGS. 1 to 3. The valve 111 may be part of the operating system, whereas the first valve 110 may be considered part of the security system of the blood treatment apparatus 100 in several embodiments. The valves 111 and 121 serve as shut-off valves that control the supply of concentrate (e.g. sodium, bicarbonate) into the circuit of the dialysis liquid system 1 during normal operation.

Such an optional valve can also be provided for the second connecting line 107, which has the reference numeral 121 and for which the statements made regarding the valve 111 may apply analogously.

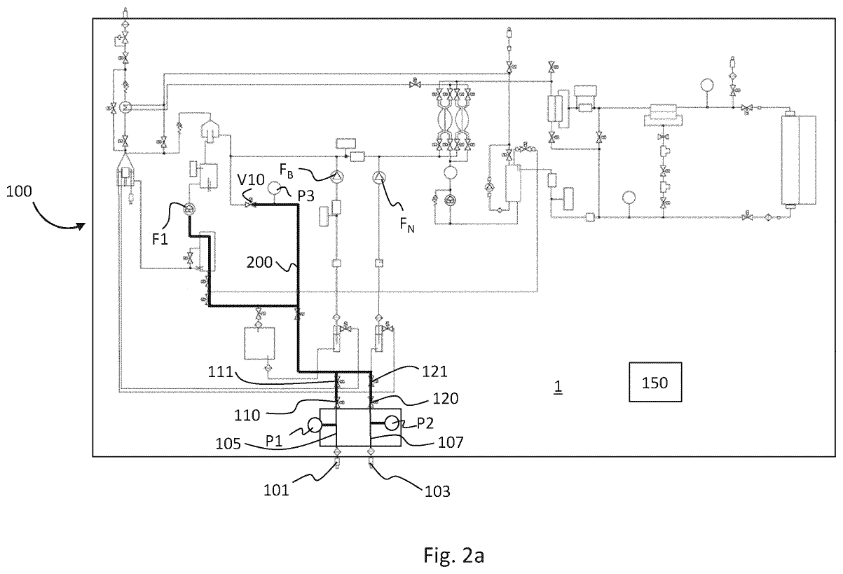

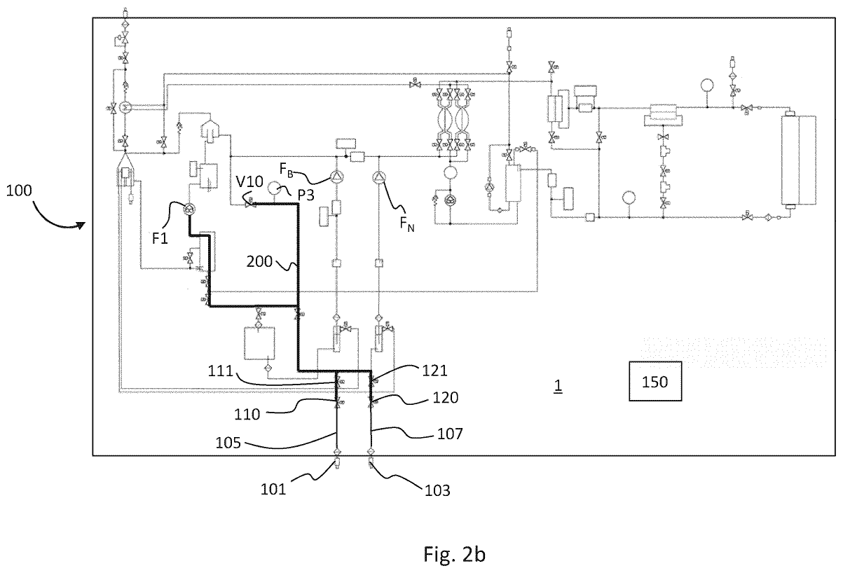

The valve V10 designated in the example of FIG. 1 and its function are described in more detail in FIG. 2a.

FIG. 2a shows the flow diagram of the dialysis liquid system 1 of FIG. 1, wherein the section 200, which is of particular importance for the tightness test or function test for which the control device or closed-loop control device 150 is programmed in several embodiments, is highlighted in bold.

Reference is made to the description of FIG. 1 in order to avoid repetition. In the following, the section 200, highlighted in the drawing and fluidically closed in FIG. 2a, will be discussed in particular.

The valve V10 in the dialysis liquid system 1 may serve, together with the valves 110, 120 and the conveying device F1 described herein, to limit and/or shut off a line volume, referred to herein as section 200. The section 200 thus possibly comprises a network of lines, in particular line sections, that are shut off but communicate with each other. Other designations, such as volume, container, receiving section for liquid, etc., would also be conceivable within the scope of the present description instead of “section”.

The conveying device F1 is arranged with respect to the section 200 such that it may convey fluid, e.g. liquid, which is present in section 200, such as RO water (reverse osmosis) introduced for checking valves, out of or into the section 200, or may effect or build up pressure and/or underpressure in section 200, wherein in certain embodiments it is prompted to do so by the control device or closed-loop control device 150.

The third pressure measuring device P3, by which e.g. a first and/or a second pressure measurement, may be carried out as described herein, is arranged in or on section 200 in order to be able to measure or detect the pressure within section 200.

FIG. 2b shows the flow diagram of the dialysis liquid system 1 of FIGS. 1 and 2a in a further embodiment in which the first and second pressure measuring devices P1 and respectively P2 have been omitted.

FIG. 3 shows schematically highly simplified the section 200 of the blood treatment apparatus 100 in a further embodiment, closed by corresponding valve switching for the purpose of tightness or function test of the—here exemplary—first valve 110.

The first connecting line 105 is shown, which is in FIG. 3 on the left connected to the concentrate supply system 140, which is roughly outlined, by the first connector 101. The optional pressure measuring device P1 is arranged in or on the connecting line 105. The first valve 110 closest to the first connector 101 may shut off or limit the section 200 toward the first connector 101 in order to measure pressure. An optional further valve 111, as detailed with regard to FIG. 1, is shown downstream of the first valve 110. By using the conveying device F1, a predetermined pressure, an overpressure or an underpressure may be generated within the section 200 as a first or as a second test pressure P+₁, P+₂, P−₁ and/or P−₂. The pressure thus generated within the section 200 may be measured by the pressure measuring device P3. The pressure prevailing meanwhile outside the section 200 may be measured by the first pressure measuring device P1. When the two pressures measured in this way are compared, a pressure gradient can be measured or determined across the first valve 110 being closed in order to test its functionality. Changes thereof can be recognized and evaluated.

The section 200 would extend starting from the conveying device F1 only until the closed optional valve 111 if, contrary to what is indicated in FIG. 3, the optional valve 111 should be tested for tightness instead of the first valve 110.

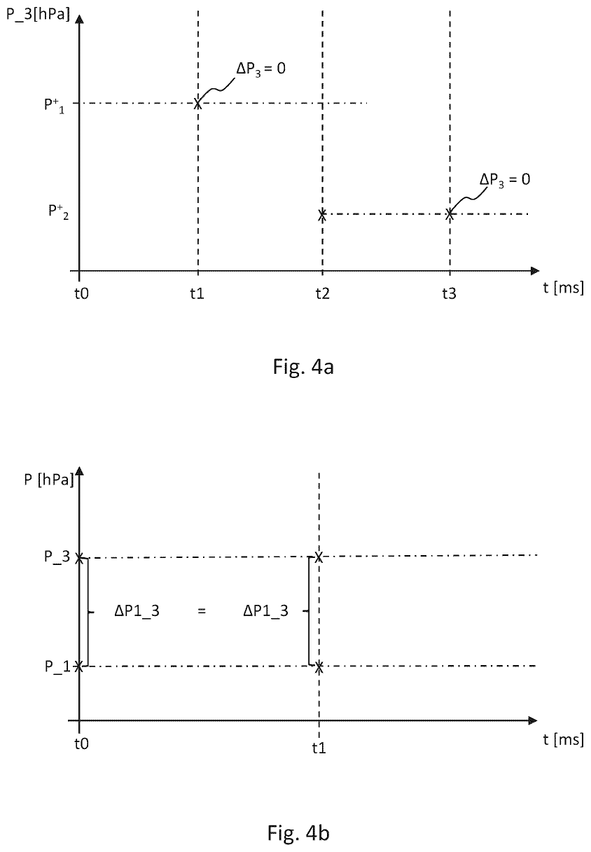

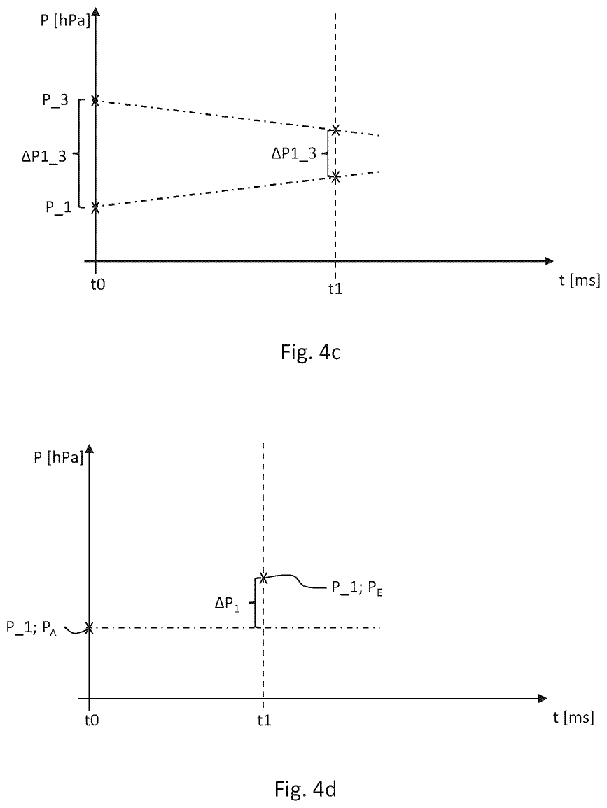

FIG. 4a shows schematically highly simplified the method step of measuring b) or measuring d), respectively, in a first way by using the blood treatment apparatus 100 as it is shown or outlined in section in e.g. FIG. 1 to FIG. 3, the reference numerals of which are referred to in the following.

The schematically highly simplified diagram shows the pressure P_3 on the y-axis, for example in the unit hectopascal [hPₐ], which is determined by the third pressure measuring device P3, over the time t on the x-axis, for example in the unit milliseconds [ms].

By the control device or closed-loop control device 150, an, preferably predetermined, overpressure P+₁ has been built up or set until the timepoint to, by actuating the conveying device F1, in the section 200 as a first test pressure which can be checked by the third pressure measuring device P3.

At the later time point t1, a measurement of the pressure P_3 takes place, referred to herein as the first pressure measurement. In the example of FIG. 4a, this is a pressure measurement done by the third pressure measuring device P3, which determines the pressure P_3 prevailing in section 200 at timepoint t1.

This pressure P_3 measured at t1, or the pressure difference ΔP₃ between P_3 at timepoint t0 and P_3 at timepoint t1, can now be evaluated using the evaluation unit. In the example of FIG. 4a, the pressure measured at timepoint t1 is equal to the pressure P_3 at timepoint to, i.e. the pressure difference observed over time is ΔP₃=0. At this point, the pressure test could be considered as passed, and the tested valve as to be functional or tight.

If the pressure P_3 or the pressure difference ΔP₃ at timepoint t1, does not meet the pressure requirements because e.g. between t0 and t1 a pressure loss occurred which does not meet or exceed e.g. predetermined criteria, threshold values, etc., an alarm or a message may be issued indicating the non-tightness of the tested valve.

In certain embodiments, the identification of the leaking or non-functional valve may be carried out, if necessary, by suitable valve switching or valve switching combinations.

In order not to run the risk that a leakage of the section 200 is not detected because the pressure of the concentrate supply system 140 or at least the pressure in the connecting lines 105, 107 happens to correspond to the first test pressure and therefore no pressure difference ΔP₃ can be observed at timepoint t1, a second test pressure P+₂ can optionally be set, e.g. at a later timepoint t2. At again a later timepoint t3, a second pressure measurement is carried out; in the example of FIG. 4a a pressure measurement by the third pressure measuring device P3, by which the pressure P_3 prevailing in section 200 is determined again at timepoint t3.

This pressure P_3 measured at timepoint t3, or the pressure difference ΔP₃ between pressure P_3 measured at timepoint t2 and pressure P_3 measured at timepoint t3, can now be evaluated using the evaluation unit. If this pressure P_3 or this pressure difference ΔP₃ determined at timepoint t3 also satisfies the pressure requirements, as shown in the example in FIG. 4a, the required tightness of the tested valve can be assumed with even greater certainty.

An analogous procedure for building up or setting a, preferably predetermined, underpressure as the first and/or second test pressure instead of the aforementioned overpressures, or a combination of an initially overpressure as the first test pressure, followed by an underpressure as the second test pressure, or vice versa is also encompassed by the present description.

FIG. 4b shows schematically highly simplified the method step of measuring b) by the blood treatment apparatus 100 in a further embodiment of its control device or closed-loop control device 150.

The schematically highly simplified diagram shows in general the pressure P on the y-axis, again over time t.

A pressure difference ΔP1_3 (i.e. P_1-P_3, or vice versa) determined at timepoint to based on a first pressure measurement by both the first pressure measuring device P1 and the third pressure measuring device P3 across these two pressure measuring devices recognizably corresponds in FIG. 4b to that pressure difference ΔP1_3 which was determined at timepoint t1 by a second pressure measurement using both the first pressure measuring device P1 and the third pressure measuring device P3. It can thus be seen that the pressure gradient across the valve being tested for tightness, here purely exemplarily, the first valve 1 0, is maintained over the period under consideration, which means that this valve can be assumed to be tight.

FIG. 4c schematically shows a pressure measurement according to the same setup as used for the pressure measurement of FIG. 4b, but with a possibly leaking first valve 110. The pressure difference ΔP1_3 at timepoint t1 is noticeably lower than at timepoint to. Depending on the specifications by or of the manufacturer, e.g. regarding threshold values, threshold ranges, limit values, etc., the tightness test of the first valve 110 may or may not be passed after evaluation.

FIG. 4d shows schematically highly simplified the method step of measuring b) using the blood treatment apparatus 100 in a further embodiment of its control device or closed-loop control device 150.

In the example of FIG. 4d, the control device or closed-loop control device 150 executes the first pressure measurement using the first pressure measuring device P1 at timepoint to. The first pressure measurement at timepoint to leads to an initial pressure P_{A} and precedes the building up or setting of the first test pressure in the closed section 200 by the conveying device F1, wherein said first test pressure is preferably predetermined but not further relevant here in terms of its level and is therefore not considered. A second pressure measurement by the pressure measuring device P1, with which the first pressure measurement was already carried out, takes place at a time after the building up or setting of the first test pressure; in the example of FIG. 4d at timepoint t1. The pressure P_1 measured thereby is then set as the final pressure PE by the control device or closed-loop control device 150.

The control device or closed-loop control device 150 is further configured to evaluate the pressure difference ΔP1 between initial pressure PA and final pressure PE by using the evaluation unit.

If there is, as indicated in FIG. 4d, a sufficiently large pressure difference ΔP1, measured against limit values or similar, it is assumed that pressurization of the tested valve from the section 200, no matter how high this was exactly, has caused an unintentional volume shift across the valve in question. The valve does not appear to be sufficiently tight at or under this pressure.

If the valve withstands this first test pressure, no matter how high it was, a test with a second test pressure may follow as described above for other embodiments.

The exact level of the first or second test pressure is not critical and can be approximated, for example, by a capacity or activity of the conveying device. Thus, the first test pressure may be built up by the conveying device conveying at a predetermined capacity, over a predetermined duration, etc. Deviating from this, the conveying device for building up the second test pressure conveys with a different, predetermined capacity over a different duration in the opposite direction, etc.

Alternatively or additionally, the first and second pressure measurements may also be carried out by the second pressure measuring device P2.

### LIST OF REFERENCE NUMERALS

1 dialysis liquid system
100 blood treatment apparatus
101 first connector
103 second connector
105 first connecting line
107 second connecting line
110 first valve
111 optional valve
120 second valve
121 optional valve
140 concentrate supply system
150 control device or closed-loop control device
200 section
F1 conveying device, here: ultrafiltration pump
F_{B} bicarbonate pump
F_{N} sodium pump
P1 first pressure measuring device
P2 second pressure measuring device
P3 third pressure measuring device
P_1 pressure measured by the pressure measuring device P1
P_3 pressure measured by the pressure measuring device P3

P⁺₁ overpressure (positive pressure) for a first pressure measurement

P⁺₂ underpressure (negative pressure)
ΔP1_3 pressure difference between two pressure measurements by the pressure measuring devices P1 and P3
ΔP₁ pressure difference between initial pressure and end pressure measured by the pressure measuring device P1
P_{A} initial pressure
P_{E} end pressure
t0, t1,
t2, t3 timepoints
V10 valve

1.-14. (canceled)

15. A blood treatment apparatus comprising:
a dialysis liquid system having devices for mixing a dialysis liquid from, or with, at least one first concentrate from a concentrate supply system;
a first connecting line arranged upstream of the dialysis liquid system and having a first connector for fluidically connecting a section of the dialysis liquid system to the concentrate supply system; and
a first valve provided downstream of the first connector in or on the first connecting line.

16. The blood treatment apparatus of claim 15, further comprising:
a first pressure measuring device for measuring a pressure prevailing in the first connecting line, wherein the first pressure measuring device is arranged between the first connector and the first valve.

17. The blood treatment apparatus of claim 15, further comprising:
a first pressure measuring device for measuring a pressure prevailing in the first connecting line, wherein the first pressure measuring device measures the pressure between the first connector and the first valve.

18. The blood treatment apparatus of claim 15, further comprising:
a second connecting line arranged upstream of the dialysis liquid system and having a second connector for fluidically connecting the dialysis liquid system to the concentrate supply system; and
a second valve which is provided downstream of the second connector in or on the second connecting line.

19. The blood treatment apparatus of claim 18, further comprising:
a second pressure measuring device for measuring a pressure prevailing in the second connecting line, wherein the second pressure measuring device is arranged between the second connector and the second valve.

20. The blood treatment apparatus of claim 18, further comprising:
a second pressure measuring device for measuring a pressure prevailing in the second connecting line, wherein the second pressure measuring device measures the pressure between the second connector and the second valve.

21. The blood treatment apparatus of claim 18, wherein the first connecting line and/or the second connecting line does not comprise a ventilation valve for establishing a connection to the atmosphere.

22. The blood treatment apparatus of claim 18, further comprising:
one or more shut-off devices, for fluidically closing the section against some or all of the remaining sections of the dialysis liquid system,
wherein the one or more shut-off devices comprise the first valve and/or the second valve.

23. The blood treatment apparatus of claim 18, further comprising:
a third pressure measuring device for measuring a pressure prevailing downstream of the first connecting line and/or downstream of the second connecting line in a section of the dialysis liquid system being in fluid communication with the first connecting line and/or the second connecting line.

24. The blood treatment apparatus of claim 23, further comprising:
an evaluation unit configured to evaluate pressures measured by the first pressure measuring device, the second pressure measuring device, and/or the third pressure measuring device or to evaluate a pressure difference;
a conveying device which is in fluid communication with the section of the dialysis liquid system, wherein the section is in fluid communication with the first connecting line or the second connecting line when the first valve or the second valve, respectively, is open;
a control device or closed-loop control device configured to prompt the following method steps:
a) building up or setting, using the conveying device, an overpressure or underpressure in the section as a first test pressure; and
b) measuring, in a first pressure measurement, the pressure prevailing in the section by using the third pressure measuring device, or measuring the pressure prevailing in the first connecting line by using the first pressure measuring device and/or measuring the pressure prevailing in the second connecting line by using the second pressure measuring device.

25. The blood treatment apparatus of claim 24, wherein the control device or closed-loop control device is further configured to carry out the first pressure measurement by using the first pressure measuring device, the second pressure measuring device, or the third pressure measuring device at a time after the building up or setting of the first test pressure in the section using the conveying device, and have the pressure, having been measured thereby, evaluated by the evaluation unit.

26. The blood treatment apparatus of claim 24, wherein the control device or closed-loop control device is further configured to carry out the first pressure measurement by using the first pressure measuring device and/or the second pressure measuring device at a time before building up or setting of the first test pressure in the section by the conveying device, and define the pressure measured thereby as initial pressure.

27. The blood treatment apparatus of claim 26, wherein the control device or closed-loop control device is further configured to carry out a second pressure measurement by using the pressure measuring device, with which the first pressure measurement was carried out, at a time after building up or setting of the first test pressure, define the pressure measured thereby as final pressure, and have a pressure difference between initial pressure and final pressure evaluated by the evaluation unit.

28. The blood treatment apparatus of claim 24, wherein the control device or closed-loop control device is further configured to prompt the following method steps:
c) building up or setting a further overpressure or underpressure as a second test pressure in the section by the conveying device;
d) measuring, in a second pressure measurement by the third pressure measuring device, the pressure prevailing in the section; and
e) evaluating, using the evaluation unit, the pressure measured in the first pressure measurement as well as in the second pressure measurement or a pressure difference between the second test pressure and the measured pressure.

29. The blood treatment apparatus of claim 28, wherein the control device or closed-loop control device is further configured to carry out the method steps from the group consisting of method steps a) to e), alternatively method steps b) and/or d), without establishing or causing a communication of the first connecting line and/or the second connecting line with the atmosphere by a corresponding valve switching.

30. The blood treatment apparatus of claim 15, wherein the blood treatment apparatus is a dialysis apparatus, hemodialysis apparatus, hemofiltration apparatus, or hemodiafiltration apparatus.

31. The blood treatment apparatus of claim 15, wherein the blood treatment apparatus is an apparatus for an acute renal replacement therapy, a chronic renal replacement therapy, or a continuous renal replacement therapy (CRRT).

32. A control device or closed-loop control device configured to prompt the following method steps:
a) building up or setting, using a conveying device of a dialysis liquid system, an overpressure or underpressure in a section of the dialysis liquid system as a first test pressure; and
b) measuring, in a first pressure measurement, a pressure prevailing in the section by using a third pressure measuring device of the dialysis liquid system, or measuring a pressure prevailing in a first connecting line by using a first pressure measuring device of the dialysis liquid system and/or measuring a pressure prevailing in a second connecting line by using a second pressure measuring device of the dialysis liquid system.

33. The control device or closed-loop control device of claim 32, wherein the control device is further configured to carry out the first pressure measurement by using the first pressure measuring device, the second pressure measuring device, or the third pressure measuring device at a time after the building up or setting of the first test pressure in the section using the conveying device, and to have the pressure, having been measured thereby, evaluated by an evaluation unit of the dialysis liquid system.
